(19) United States
(12) Patent Application Publication
(10) Pub. No.: US 2025/0246288 A1
(43) Pub. Date: Jul. 31, 2025

(54) METHODS AND APPARATUS FOR RECOMMENDING ESCALATION FOR A MECHANICAL CIRCULATORY SUPPORT DEVICE

(71) Applicant: Abiomed, Inc., Danvers, MA (US)

(72) Inventors: Elise Jortberg, Danvers, MA (US); Erik Kroeker, Danvers, MA (US); Samantha Polak, Danvers, MA (US)

(73) Assignee: Abiomed, Inc., Danvers, MA (US)

(21) Appl. No.: 19/036,913

(22) Filed: Jan. 24, 2025

Related U.S. Application Data
(60) Provisional application No. 63/625,656, filed on Jan. 26, 2024.

(51) Int. Cl. G16H 20/40; A61M 60/122; A61M 60/508; A61M 60/585; G16H 10/60; G16H 50/70

(52) U.S. Cl. CPC G16H 20/40; A61M 60/122; A61M 60/508; A61M 60/585; G16H 10/60; G16H 50/70; A61M 2205/04; A61M 2205/3303; A61M 2205/3327; A61M 2205/3331; A61M 2205/3365; A61M 2205/3561; A61M 2205/502; A61M 2210/125; A61M 2230/04

(57) ABSTRACT

Methods and apparatus for determining an escalation recommendation for a patient having an implanted mechanical circulatory support device are provided. The method includes receiving, via a user interface associated with a mechanical circulatory support device, an indication to determine an escalation recommendation for a patient, determining values for a set of features, wherein the set of features includes one or more first features associated with the mechanical circulatory support device and one or more second features associated with the patient, providing the values for the set of features as input to a trained model to generate a model output, and displaying on the user interface, an escalation recommendation for the patient based, at least in part, on the model output.

### RELATED APPLICATIONS

This application claims the benefit under 35 U.S.C. § 119(e) to U.S. Provisional Patent Application No. 63/625,656, filed Jan. 26, 2024, and titled, “METHODS AND APPARATUS FOR RECOMMENDING ESCALATION FOR A MECHANICAL CIRCULATORY SUPPORT DEVICE,” the entire contents of which is hereby incorporated by reference herein.

### TECHNICAL FIELD

Embodiments disclosed herein relate to medical devices, such as catheters and catheter based devices.

### BACKGROUND

Blood pump assemblies, such as intracardiac or intravascular blood pumps may be introduced in the heart to deliver blood from the heart into an artery. Such mechanical circulatory support devices are often introduced to support the function of the heart after a patient suffers a cardiac episode. One such class of devices is the set of devices known as the “Impella” heart pump. Some blood pump assemblies may be introduced percutaneously through the vascular system during a cardiac procedure. Specifically, blood pump assemblies can be inserted via a catheterization procedure through the femoral artery or the axillary/subclavian artery, into the ascending aorta, across the valve and into the left ventricle. The inserted blood pump assembly may be configured to pull blood from the left ventricle of the heart through a cannula and expel the blood into the aorta. A blood pump assembly may also be configured to pull blood from the inferior vena cava and to expel blood into the pulmonary artery. Some mechanical circulatory support devices are powered by an on-board motor, while others are powered by an external motor and a drive cable.

### SUMMARY

Some patients experiencing heart failure may receive support from a mechanical circulatory support (MCS) device. During support, patients may require more support than certain types of MCS devices can provide. The decision of when and how to escalate patient support may be a multi-faceted decision.

The systems, devices, and methods described herein relate to a data-driven technique that provides context to the decision on when and how to escalate patient support from a first MCS device to a second MCS device. For instance, some embodiments of the present disclosure relate to a clinical decision support tool configured to display a recommendation to escalate patient support based, at least in part, on metrics determined from MCS device signals sensed by one or more sensors on the MCS device and a model trained on historical patient cohort data.

In some embodiments, a computer-implemented method of determining an escalation recommendation for a patient having an implanted mechanical circulatory support device is provided. The method includes receiving, via a user interface associated with a mechanical circulatory support device, an indication to determine an escalation recommendation for a patient, determining values for a set of features, wherein the set of features includes one or more first features associated with the mechanical circulatory support device and one or more second features associated with the patient, providing the values for the set of features as input to a trained model to generate a model output, and displaying on the user interface, an escalation recommendation for the patient based, at least in part, on the model output.

In one aspect, the user interface associated with the mechanical circulatory support device is displayed by a controller of the mechanical circulatory support device. In another aspect, the user interface associated with the mechanical circulatory support device is displayed by a computing device communicatively coupled to the mechanical circulatory support device. In another aspect, the one or more first features associated with the mechanical circulatory support device include a feature associated with operation of the mechanical circulatory support device. In another aspect, the feature associated with operation of the mechanical circulatory support device includes one or more of motor current, pressure information, pump speed or blood flow. In another aspect, the one or more second features associated with the patient include one or more patient physiological features. In another aspect, the one or more patient physiological features include one or more of left ventricular end diastolic pressure, heart rate, pulsatility, contractility, mean arterial pressure, ejection fraction, or cardiac output. In another aspect, the one or more second features associated with the patient include one or more features derived from an electronic health record associated with the patient. In another aspect, at least one value of the values in the set of features is a derived value determined over a particular time window. In another aspect, at least one value of the values in the set of features is a measure of variability determined over a particular time window.

In another aspect, the trained model is a model trained on historical patient cohort data associated with patients that have undergone escalation from a first type of mechanical circulatory support device to a second type of mechanical circulatory support device, the second type of mechanical circulatory support device having a higher maximum output than the first type of mechanical circulatory support device. In another aspect, displaying on the user interface, an escalation recommendation for the patient based, at least in part, on the model output comprises displaying the escalation recommendation during performance of a medical procedure on the patient. In another aspect, the medical procedure comprises a percutaneous coronary intervention procedure. In another aspect, the method further includes tracking values for the set of features over time during performance of a medical procedure, and updating the escalation recommendation for the patient displayed on the user interface during the medical procedure based on the tracked values. In another aspect, the trained model comprises a machine learning model.

In some embodiments, a mechanical circulatory support system is provided. The mechanical circulatory support system includes a heart pump including at least one sensor configured to sense operation data of the heart pump, and a controller. The controller is configured to determine values for a set of features, wherein the set of features includes one or more first features associated with mechanical circulatory support device and one or more second features associated with a patient, wherein the values for the one or more first features are determined based, at least in part, on the operation data of the heart pump, provide the values for the set of features as input to a trained model to generate a model output, and display on a user interface associated with the mechanical circulatory support system, an escalation recommendation for the patient based, at least in part, on the model output.

In one aspect, the controller includes a display, and wherein the user interface is displayed on the display of the controller. In another aspect, the controller is configured to display on a user interface associated with the mechanical circulatory support system, an escalation recommendation for the patient by transmitting an indication of the escalation recommendation to a computing device communicatively coupled to the controller, wherein the computing device is configured to display the user interface. In another aspect, the one or more first features includes one or more of motor current, pressure information, pump speed or blood flow. In another aspect, the one or more second features associated with the patient include one or more patient physiological features. In another aspect, the one or more patient physiological features include one or more of left ventricular end diastolic pressure, heart rate, pulsatility, contractility, mean arterial pressure, ejection fraction, or cardiac output. In another aspect, the one or more second features associated with the patient include one or more features derived from an electronic health record associated with the patient. In another aspect, at least one value of the values in the set of features is a derived value determined over a particular time window. In another aspect, at least one value of the values in the set of features is a measure of variability determined over a particular time window. In another aspect, the trained model is a model trained on historical patient cohort data associated with patients that have undergone escalation from a first type of mechanical circulatory support device to a second type of mechanical circulatory support device, the second type of mechanical circulatory support device having a higher maximum output than the first type of mechanical circulatory support device.

In some embodiments, a method of training a model to output an escalation recommendation for a patient having an implanted mechanical circulatory support device is provided. The method includes receiving historical patient cohort data for a plurality of patients that have undergone escalation from a first type of mechanical circulatory support device to a second type of mechanical circulatory support device, the second type of mechanical circulatory support device having a higher maximum output than the first type of mechanical circulatory support device, associating an escalation label with each patient in the historical patient cohort data to generate labeled data, training a machine learning model based on the labeled data to generate a trained model for outputting an escalation recommendation, and outputting the trained model.

In one aspect, the historical patient cohort data includes values for one or more features captured during a period of support provided by the first type of mechanical circulatory support device and/or values for one or more features captured during a period of support provided by the second type of mechanical circulatory support device. In another aspect, the historical patient cohort data includes, for each of the plurality of patients, values for a set of features, wherein the set of features includes one or more first features associated with the first type of mechanical circulatory support device and/or the second type of mechanical circulatory support device and one or more second features associated with the patient. In another aspect, the one or more first features associated with the first type of mechanical circulatory support device and/or the second type of mechanical circulatory support device include a feature associated with operation of the first type of mechanical circulatory support device and/or the second type of mechanical circulatory support device. In another aspect, the feature associated with operation of the first type of mechanical circulatory support device and/or the second type of mechanical circulatory support device includes one or more of motor current, pressure information, pump speed or blood flow. In another aspect, the one or more second features associated with the patient include one or more patient physiological features. In another aspect, the one or more patient physiological features include one or more of left ventricular end diastolic pressure, heart rate, pulsatility, contractility, mean arterial pressure, ejection fraction, or cardiac output. In another aspect, the one or more second features associated with the patient include one or more features derived from an electronic health record associated with the patient.

In another aspect, at least one value of the values in the set of features is a derived value determined over a particular time window. In another aspect, at least one value of the values in the set of features is a measure of variability determined over a particular time window. In another aspect, at least some of the values for the set of features are extracted at particular time intervals during a period of support for the patient. In another aspect, the period of support comprises a period of support during which support was provided to the patient by the first type of mechanical circulatory support device. In another aspect, the period of support comprises a period of support during which support was provided to the patient by the second type of mechanical circulatory support device.

### BRIEF DESCRIPTION OF DRAWINGS

The foregoing and other objects and advantages will be apparent upon consideration of the following detailed description, taken in conjunction with the accompanying drawings, in which like reference characters refer to like parts throughout, and in which:

FIG. 1 illustrates a flowchart of a process for training a model to output an escalation recommendation for a patient, in accordance with some embodiments of the present technology;

FIG. 2 illustrates a flowchart of a process for using a trained model to determine an escalation recommendation for a patient, in accordance with some embodiments of the present technology; and

FIG. 3 illustrates an exemplary patient console in accordance with some embodiments of the present technology.

### DETAILED DESCRIPTION

As it is known, mechanical circulatory support (MCS) devices may provide a level of support for a patient. For example, the Impella CP® device provided by Abiomed, Inc. (Danvers, MA) provides up to 4.3 Liters/minute of support while the Impella 5.5® device provided by Abiomed, Inc. (Danvers, MA) provides up to 6 Liters/minute of support. A patient with an implanted MCS device may sometimes need more support than the implanted MCS device can provide. To provide the additional support, a physicians may decide to replace the implanted MCS device with an MCS device that can provide greater levels of support. Determining if and when a patient may require escalation to a MCS device that can provide greater support may be a multi-faceted decision based on a variety of factors including, but not limited to, the observations of the physician. The inventors have recognized and appreciated that patients and/or healthcare providers may benefit from a data-driven technique that may predict when a patient would benefit from escalation to an MCS device that can provide greater support than their currently implanted MCS device.

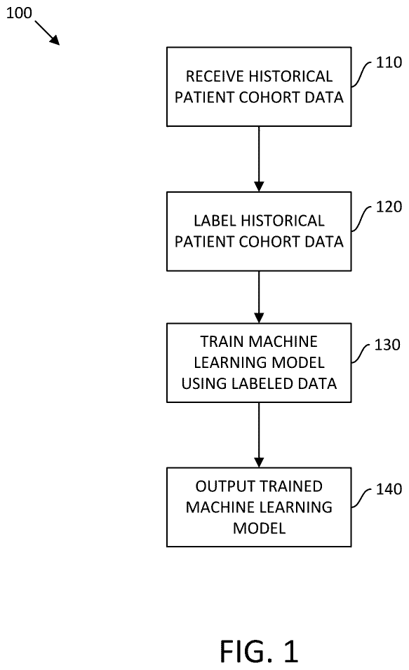

FIG. 1 illustrates a flowchart of a process 100 for training a model to output a recommendation to escalate a patient from a first type of MCS device support to a second type of MCS device support, in accordance with some embodiments of the present technology. Process 100 begins in act 110, where historical patient cohort data is received for a plurality of patients. In some embodiments, the plurality of patients includes patients that have undergone escalation from a first type of MCS device support to a second type of MCS device support. For example, in some embodiments, the plurality of patients may be patients that have undergone escalation from Impella CP® support to Impella 5.5® support. In other embodiments, the plurality of patients may be patients that have undergone escalation from Impella CP® support to any other suitable type of MCS device that can provide between about 4.4 L/min of support and about 6 L/min of support. As will be appreciated, the plurality of patients may include patients that have undergone escalation from a first MCS device to a second MCS device which has a maximum blood flow rate greater than the maximum blood flow rate of the first MCS device. In some embodiments, the plurality of patients may include patients having MCS device support that did not undergo escalation to a different type of MCS device support. In some embodiments, the historical patient cohort data may include values for one or more features captured during a period of support provided by a first MCS device and/or values for one or more features captured during a period of support provided by a second MCS device. In other embodiments, the historical patient cohort data may include only values for one or more features captured during a period of support provided by a first MCS device before escalation to a second MCS device. Examples of features that may be included in the historical patient cohort data include any suitable hemodynamic parameter and/or pump parameter. For example, such features may include, but are not limited to, heart pump operation features (e.g., motor current, pressure information, pump speed, blood flow) and patient physiological features (e.g., left ventricular end diastolic pressure (LVEDP), heart rate, pulsatility, contractility, mean arterial pressure (MAP), ejection fraction, cardiac output). In some embodiments, other features (e.g. features derived from a patient's electronic health record) may also be included in the historical patient cohort data. In some embodiments, features included in the historical patient cohort data may include medication information associated with one or more of the plurality of patients.

After historical patient cohort data is received in act 110, process 100 may proceed to act 120, where the received historical data is labeled. As will be appreciated, the received historical data may be labeled according to different scenarios. For example, in one scenario, the patient receiving support from an Impella CP® device may be escalated to receive support from an Impella 5.5® device. In another example, in a second scenario, the patient receiving support from an Impella ECP® device may be escalated to receive support from an Impella 5.5® device. In some embodiments, the received historical data may be labeled based on the level of a first type of MCS device support and the level of a second type of MCS device support. As will be appreciated, the received historical data may be labeled according to any suitable method.

After historical data for each of the patients included in the received historical patient data is labeled in act 120 as described herein, process 100 may proceed to act 130, where a model (e.g., a machine learning model) is trained using the labeled data. For instance, values for a plurality of features extracted at particular time intervals (e.g., 5 second intervals, 15 second intervals, 30 second intervals) during a period of support for each patient in the historical patient cohort may be provided as input to the model, and the model may be trained to output the associated label for the patient. As will be appreciated, the period of support may be a period during which support is provided by the first MCS device. The period of support may alternatively be a period during which support is provided by the second MCS device. The values for the features may be values at a single point in time, derived values (e.g., median or mean values) determined over a particular time window, and/or measures of variability (e.g., standard deviation) of the values for a particular time window.

When trained on a large amount of data included in the historical patient cohort, the model may learn (e.g., by changing weights in the model) characteristics of the features values that predict the corresponding labels for the patients from the labeled data. In some embodiments, the model is a classification model. It should be appreciated that in such embodiments, any suitable classification model may be used, examples, of which include a neural network (e.g., configured to implement deep learning techniques) based model, a random forest classifier model, a decision trees model (e.g., a gradient boosting decision trees model), or a logistic regression model. Process 100 then proceeds to act 140, where the trained model is output for use in predicting escalation for patients not included in the historical patient cohort data.

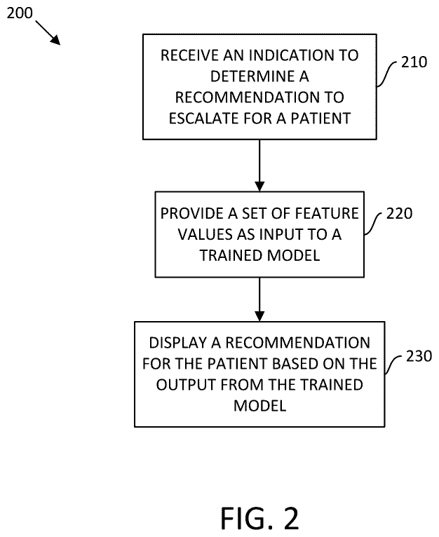

FIG. 2 is a flowchart of a process 200 for determining a recommendation to escalate support associated with a patient having a first MCS device. Process 200 begins in act 210, where an indication is received to determine if an escalation is recommended from a first MCS device to a second MCS device for a patient. It should be appreciated that a determination of an escalation recommendation for a patient using one or more of the techniques described herein may be performed at any suitable time. For instance, in some embodiments, the escalation recommendation may be determined during a period of support from the first MCS device and prior to escalation, to determine when to initiate escalation.

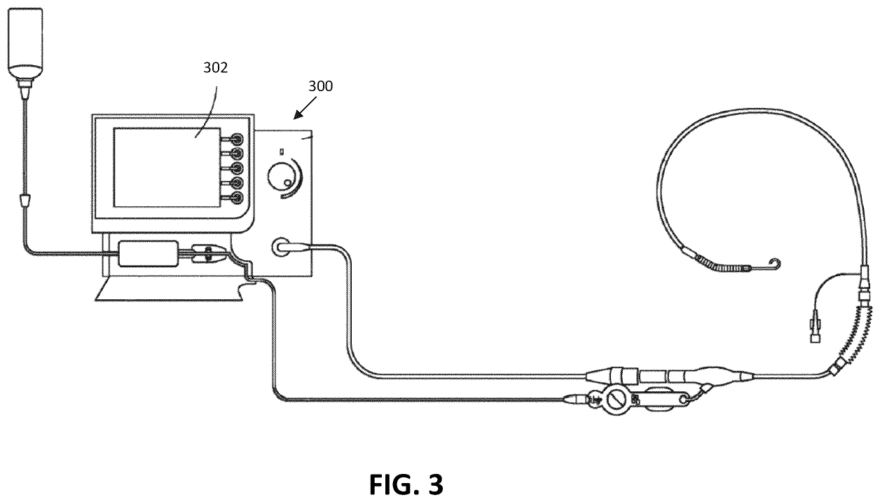

FIG. 3 illustrates an example of a portion of a patient console 300 configured to display a user interface with which a user (e.g., a healthcare provider) may interact to view a recommendation to escalate. For instance, information associated with pump operation and physiological parameters associated with the patient within whom an MCS device is implanted may be displayed on the user interface. In the example patient console 300, the display 302 may include different widgets for displaying different types of data including pump speed (e.g. P-level), the patient's heart rate, and other metrics such as patient hemodynamic parameters. Some widgets may be configured to display trend information for various metrics over time, such as cardiac output (CO), aortic pressure (AoP), and LVEDP. One or more of the displayed (or not displayed) metrics may be used to determine an escalation recommendation of the patient according to the techniques described herein. Additionally, in some embodiments, one or more metrics from the patient's electronic health record (EHR) may be used to determine the escalation recommendation. In some embodiments, information based, at least in part, on the patient's determined escalation recommendation may automatically be stored in the patient's EHR.

The patient console 300 may include instructions on the display 302 to start the escalation process. In some embodiments, the patient console 300 may instead include a recommendation to escalate to a greater level of support. In some embodiments, the escalation recommendation may include a recommendation of the flow rate (e.g., 6 L/min) and/or a recommendation of a type of MCS device (e.g., Impella 5.5®). As will be appreciated, any suitable second MCS device may be used when escalating to a greater level of support.

In some embodiments, the display 302 may be provided on a mobile device of a user. For example, the mobile device may be coupled to the patient console 300 via one or more networks. The user interface displayed on a mobile device may be the same or different as what is displayed on the patient console 300.

Returning to process 200, in response to receiving an indication to determine an escalation recommendation, process 200 may proceed to act 220, where a set of feature values may be provided as input to a trained model (e.g., a machine learning model trained in accordance with the techniques described herein). For example, a user may interact with a user interface element displayed on a user interface (e.g., display 302), and in response, a plurality of values for features associated with the patient and/or the pump may be determined and provided as input to the trained model. In some embodiments, a same set of features used to train the model may be determined and provided as input to the trained model in act 220. In other embodiments, a subset of the set of features used to train the model (e.g., only those features most predictive of the need to escalate) may be determined and provided as input to the trained model in act 220. In some embodiments, one or more features not used to train the model (e.g., one or more values received from the patient's EHR) may also be used to determine an escalation recommendation for the patient. In some embodiments, the display 302 may be configured to display an indication that an escalation recommendation is being calculated based on the set of feature values provided as input to the trained model, in accordance with some embodiments of the present technology.

After providing the set of feature values as input to the trained model, process 200 may proceed to act 230, where a recommendation to escalate of the patient may be displayed on the display 302, wherein the escalation recommendation is determined based, at least in part, on an output of the trained model. A healthcare provider viewing the display 302 may use the escalation recommendation to guide decisions about whether, when and/or how to escalate the patient to a second MCS device. In this way, some embodiments of the present technology may be used as a clinical decision support tool to facilitate decision making regarding escalating a patient to a second MCS device.

In some embodiments, an escalation recommendation may be tracked over time. For example, the escalation recommendation may be tracked during a percutaneous coronary intervention (PCI) procedure to identify if the patient needs greater support at a point during the procedure. In another example, the escalation recommendation may be tracked during a period of support for a patient experiencing heart failure. For example, an escalation recommendation may be tracked for patients experiencing cardiogenic shock to determine whether such patients who may be using an MCS device to provide cardiac support should be escalated to a different type of MCS device configure to have a greater maximum flow rate.

It should be appreciated that any of the user interfaces described herein may be displayed on a display of a controller associated with the MCS device and/or may be displayed on an auxiliary display coupled to a controller of the MCS device, for example, over one or more networks.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The above-described embodiments of the present technology can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as a controller that controls the above-described function. A controller can be implemented in numerous ways, such as with dedicated hardware, or with general purpose hardware (e.g., one or more processor) that is programmed using microcode or software to perform the functions recited above, and may be implemented in a combination of ways when the controller corresponds to multiple components of a system.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

In the claims, as well as in the specification above, all transitional phrases such as “comprising,” “including”, “carrying”, “having”, “containing”, “involving”, “holding”, “composed of”, and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A computer-implemented method of determining an escalation recommendation for a patient having an implanted mechanical circulatory support device, the method comprising:
receiving, via a user interface associated with a mechanical circulatory support device, an indication to determine an escalation recommendation for a patient;
determining values for a set of features, wherein the set of features includes one or more first features associated with the mechanical circulatory support device and one or more second features associated with the patient;
providing the values for the set of features as input to a trained model to generate a model output; and
displaying on the user interface, an escalation recommendation for the patient based, at least in part, on the model output.

2. The method of claim 1, wherein the user interface associated with the mechanical circulatory support device is displayed by a controller of the mechanical circulatory support device.

3. The method of claim 1, wherein the user interface associated with the mechanical circulatory support device is displayed by a computing device communicatively coupled to the mechanical circulatory support device.

4. The method of claim 1, wherein the one or more first features associated with the mechanical circulatory support device include a feature associated with operation of the mechanical circulatory support device.

5. The method of claim 4, wherein the feature associated with operation of the mechanical circulatory support device includes one or more of motor current, pressure information, pump speed or blood flow.

6. The method of claim 1, wherein the one or more second features associated with the patient include one or more patient physiological features.

7. The method of claim 6, wherein the one or more patient physiological features include one or more of left ventricular end diastolic pressure, heart rate, pulsatility, contractility, mean arterial pressure, ejection fraction, or cardiac output.

8. The method of claim 1, wherein the one or more second features associated with the patient include one or more features derived from an electronic health record associated with the patient.

9. The method of claim 1, wherein at least one value of the values in the set of features is a derived value determined over a particular time window.

10. The method of claim 1, wherein at least one value of the values in the set of features is a measure of variability determined over a particular time window.

11. The method of claim 1, wherein the trained model is a model trained on historical patient cohort data associated with patients that have undergone escalation from a first type of mechanical circulatory support device to a second type of mechanical circulatory support device, the second type of mechanical circulatory support device having a higher maximum output than the first type of mechanical circulatory support device.

12. The method of claim 1, wherein displaying on the user interface, an escalation recommendation for the patient based, at least in part, on the model output comprises displaying the escalation recommendation during performance of a medical procedure on the patient.

13. The method of claim 12, wherein the medical procedure comprises a percutaneous coronary intervention procedure.

14. The method of claim 1, further comprising:
tracking values for the set of features over time during performance of a medical procedure; and
updating the escalation recommendation for the patient displayed on the user interface during the medical procedure based on the tracked values.

15. The method of claim 1, wherein the trained model comprises a machine learning model.

16. A mechanical circulatory support system, comprising:
a heart pump including at least one sensor configured to sense operation data of the heart pump; and
a controller configured to:
determine values for a set of features, wherein the set of features includes one or more first features associated with mechanical circulatory support device and one or more second features associated with a patient, wherein the values for the one or more first features are determined based, at least in part, on the operation data of the heart pump;
provide the values for the set of features as input to a trained model to generate a model output; and
display on a user interface associated with the mechanical circulatory support system, an escalation recommendation for the patient based, at least in part, on the model output.

17. (canceled)

18. The mechanical circulatory support system of claim 16, wherein the controller is configured to display on a user interface associated with the mechanical circulatory support system, an escalation recommendation for the patient by transmitting an indication of the escalation recommendation to a computing device communicatively coupled to the controller, wherein the computing device is configured to display the user interface.

19.-25. (canceled)

26. A method of training a model to output an escalation recommendation for a patient having an implanted mechanical circulatory support device, the method comprising:
receiving historical patient cohort data for a plurality of patients that have undergone escalation from a first type of mechanical circulatory support device to a second type of mechanical circulatory support device, the second type of mechanical circulatory support device having a higher maximum output than the first type of mechanical circulatory support device;
associating an escalation label with each patient in the historical patient cohort data to generate labeled data;
training a machine learning model based on the labeled data to generate a trained model for outputting an escalation recommendation; and
outputting the trained model.

27. The method of claim 26, wherein the historical patient cohort data includes values for one or more features captured during a period of support provided by the first type of mechanical circulatory support device and/or values for one or more features captured during a period of support provided by the second type of mechanical circulatory support device.

28. The method of claim 26, wherein the historical patient cohort data includes, for each of the plurality of patients, values for a set of features, wherein the set of features includes one or more first features associated with the first type of mechanical circulatory support device and/or the second type of mechanical circulatory support device and one or more second features associated with the patient.

29.-38. (canceled)
